(19) United States
(12) Patent Application Publication
(10) Pub. No.: US 2026/0166544 A1
(43) Pub. Date: Jun. 18, 2026

(54) Microcalorimetry for High-Throughput Screening of Bioenergetics

(71) Applicant: President and Fellows of Harvard College, Cambridge, MA (US)

(72) Inventors: Juanjuan Zheng, Somerville, MA (US); Joost Vlassak, Lexington, MA (US); Daniel Needleman, Somerville, MA (US)

(21) Appl. No.: 19/127,508

(22) PCT Filed: Nov. 9, 2023
(86) PCT No.: PCT/US2023/079180

§ 371 Date: May 6, 2025

Related U.S. Application Data
(60) Provisional application No. 63/423,954, filed on Nov. 9, 2022.

(51) Int. Cl. B01L 3/00; G01N 27/327; G01N 27/447

(52) U.S. Cl. CPC B01L 3/502707; B01L 3/5027; G01N 27/3274; G01N 27/44782; A61K 2300/00; B01L 2300/1827

(57) ABSTRACT

A calorimetric sensor for high-throughput screening of bioenergetics, including single cells, includes a substrate defining an interior cavity; a thermally and electrically insulating membrane extending from the substrate across the interior cavity; a plurality of thermally conductive strips bonded to the membrane; a first thermopile on the membrane and comprising a plurality of thermocouples connected in series. The thermocouples of the first thermopile extend between the first thermally conductive strip and the second thermally conductive strip, providing thermal communication therebetween, and are configured to provide a measurement of a temperature difference between the first and second thermally conductive strips. A thermally and electrically insulating coating covers the thermally and electrically insulating membrane, the thermally conductive strips, and the first thermopile. A sample capillary and a first reference capillary are adhered to the thermally and electrically insulating coating opposite respective thermally conductive strips.

### GOVERNMENT SUPPORT

This invention was made with government support under Grant No. 2011754 awarded by the National Science Foundation (NSF). The U.S. Government has certain rights in the invention.

### BACKGROUND

The discussion of the background state of the art, below, may reflect hindsight gained from the disclosed invention(s); and these characterizations are not necessarily admitted to be prior art.

Calorimetry, which measures the heat generated from a sample, is used to provide a direct measure of bioenergetics, such as metabolic rates. Calorimetry, however, is rarely used in cell and developmental biology due to limitations in sensitivity and throughput.

### SUMMARY

A calorimetric sensor and a method for performing calorimetry on a biological sample are described herein, where various embodiments of the apparatus and methods may include some or all of the elements, features, and steps described below.

A calorimetric sensor for high-throughput screening of bioenergetics, including single cells, includes a substrate defining an interior cavity; a thermally and electrically insulating membrane extending from the substrate across the interior cavity; a plurality of thermally conductive strips bonded to the thermally insulating membrane; a first thermopile on the thermally and electrically insulating membrane and comprising a plurality of thermocouples connected in series. The thermocouples of the first thermopile extend between the first thermally conductive strip and the second thermally conductive strip and provide thermal communication therebetween and are configured to provide a measurement of a temperature difference between the first and second thermally conductive strips. A thermally and electrically insulating coating covers the thermally and electrically insulating membrane, the thermally conductive strips, and the first thermopile; these components, together, form a sensing assembly. A sample capillary with a hydrophilic surface is adhered to the thermally and electrically insulating coating opposite a first of the thermally conductive strips via application of water at an interface of the sample capillary and the thermally and electrically insulating coating and via capillary action during evaporation of the water. A first reference capillary with a hydrophilic surface is adhered to the thermally and electrically insulating coating opposite a second of the thermally conductive strips via application of water at an interface of the reference capillary and the thermally and electrically insulating coating and via capillary action during evaporation of the water. Advantageously, the calorimetric sensor further includes a second reference capillary and a second thermopile. The second reference capillary has a hydrophilic surface and is adhered to the thermally and electrically insulating coating opposite a third of the thermally conductive strips via application of water at an interface of the reference capillary and the thermally and electrically insulating coating and via capillary action during evaporation of the water. The second thermopile is positioned on the thermally and electrically insulating membrane on an opposite side of the first thermally conductive strip from the first thermopile. The second thermopile also includes a plurality of thermocouples connected in series, and the thermocouples of the second thermopile extend between the first thermally conductive strip and the third thermally conductive strip and are configured to provide a measurement of a temperature difference between the first and third thermally conductive strips.

A method for performing calorimetry on a biological sample using the calorimetric sensor includes passing a biological sample through the sample capillary and filling the first reference capillary with a reference fluid. The first thermopile is used to measure a temperature difference from the first conductive strip to the second conductive strip. A bioenergetic level of the biological sample is determined based on the measurement of the temperature difference from the first conductive strip to the second conductive strip.

A method for fabricating a calorimetric sensor for high-throughput screening of bioenergetics includes depositing a thermally and electrically insulating membrane onto a substrate. A central region of the substrate is etched away to create an interior cavity. At least one thermopile is formed on the thermally and electrically insulating membrane. A plurality of thermally conductive strips are deposited on the thermally and electrically insulating membrane, wherein each thermally conductive strip is in thermal contact with at least one of the thermopiles. A thermally and electrically insulating coating is deposited on the thermally and electrically insulating membrane, the thermally conductive strips, and the at least one thermopile, wherein these components, together, form a sensing assembly. A surface treatment is provided to a sample capillary and at least one reference capillary to give the capillaries hydrophilic surfaces and to the thermally and electrically insulating coating to give the thermally and electrically insulating coating a hydrophilic surface. The sample capillary and the at least one reference capillary are then placed in contact with the thermally and electrically insulating coating opposite respective thermally conductive strips. Water is then applied at interfaces of the capillaries and the thermally and electrically insulating coating, and the water is evaporated to adhere the capillaries to the thermally and electrically insulating coating via capillary action.

Described herein is a microfabricated calorimetric sensor that allows high-throughput measurements of bioenergetics with single-cell sensitivity. The microfabricated calorimetric sensor can overcome the sensitivity and throughput limitations of previous calorimeters, thus enabling diverse calorimetric studies of metabolic rate in cell biology and developmental biology.

The calorimetric sensor described herein can provide a near order-of-magnitude increase in sensitivity above current state-of-the-art calorimeters, from 200 pW to 31 pW. Since the average mammalian cell has a total heat production rate of ˜60 pW, this improvement will push calorimetry into a regime where it can be used for single-cell measurements on a broad range of mammalian cell types. This sensitivity will enable direct measurements of cell-to-cell metabolic heterogeneity, which has remained poorly characterized despite being hypothesized to play crucial roles in determining variations in cell developmental fate and cell signal processing, as well as in cancer progression and response to drugs.

The calorimetric sensor described herein can also have orders-of-magnitude-higher throughput than existing calorimeters. This improvement will enable high-throughput screens of metabolic rate. Because the growth of cancer cells is strongly dependent on their metabolic state and because the response of bacteria to antibiotics is linked to their metabolism, calorimetric-based screens for small molecules that alter the metabolic rates of cancer cells and bacteria can help to identify compounds that are both useful for scientifical research and medically beneficial.

### BRIEF DESCRIPTION OF THE DRAWINGS

FIG. 1 is a perspective view of a calorimetric sensor 10 with superior resolution achieved through a high-sensitivity nanofabricated thermopile 12 coupled to a set of capillaries 14, 15 and a low-heat loss environment.

FIG. 2 is an exploded view of the calorimetric sensor 10 of FIG. 1

FIG. 3 is a sectional view of the calorimetric sensor 10 from the section 3 of the calorimetric sensor 10 illustrated in FIG. 1

FIG. 4 is an enlarged view of the thermopiles 12 and tungsten heater 30 at the center of the section 27 of the calorimetric sensor shown in FIG. 2.

FIG. 5 is a schematic illustration of a finite element model of a calorimetric sensor 10.

FIG. 6 is a thermal model of the temperature distribution in a calorimetric sensor for a distributed biological sample, where the indicator temperature shading represents the temperature difference between the local temperature and the ambient temperature, wherein the darkening center represents the highest temperature differences (4-4.5 μK), with the lowest temperature differences are represented by the darkening at the perimeter (approaching 0 μK).

FIG. 7 is a thermal model of the temperature distribution in a calorimetric sensor for a small biological sample.

FIG. 8 is a plot of noise equivalent power (NEP) as a function of the width [i.e., the number of thermocouples (n)] of each thermopile and the length of each thermopile in a calorimetric sensor.

FIG. 9 is a plot of NEP as a function of thickness of the thermopile for a 2.5-mm long thermopile consisting of 126 thermocouples (n=126).

FIG. 10 plots the volumetric sensitivity of a calorimetric sensor as a function of capillary dimensions.

FIGS. 11-18 provide side cross-sectional views of the component layers, including silicon 16 with (100) crystal orientation and LPCVD Si₃N₄ 26, of a calorimetric sensor 10 at sequential stages of fabrication. The contact pads are not shown.

FIG. 19 is a top-side optical image of a fabricated calorimetric sensor 10.

FIG. 20 is a side-view optical image of a calorimetric sensor 10 mounted in a vacuum chamber 38.

FIG. 21 is a perspective view of the calorimetric sensor 10 assembly on a sensor stage 40.

FIG. 22 is a schematic illustration of an entire data acquisition apparatus 43 and measurement setup including a calorimetric sensor 10 mounted in a vacuum chamber 38.

FIG. 23 is a schematic illustration of calibration of a calorimetric sensor 10.

FIG. 24 is a plot of the voltage response of a calorimetric sensor with a sensitivity 120 μV/μW as a function of the power applied to the heating element.

FIG. 25 is a plot of voltage as a function of power for a sensor, which offers a measurement of the response time constant.

FIG. 26 is a plot of power as a function of time, which demonstrates the response of the sensor over a period of approximately 10 hours without power applied to the sensor heating element. The plotted half difference 78 of the voltage output of the two colinear thermopiles (V_{TP1} 64 and V_{TP2} 66) represents the sensor signal and is evidently much less sensitive to thermal drift (˜40 pw) than the voltage signal 64 and 66 from individual thermopiles. The average 76 of the output of the two colinear thermopiles (V_{TP1} 64 and V_{TP2} 66) is also plotted.

FIG. 27 is a top-side view of a calorimetric sensor 10 mounted in a sensor carrier 42.

FIG. 28 is a top-side view of a printed circuit board (PCB) 82 mounted atop the sensing carrier 42 and the calorimetric sensor 10, wherein the sensing carrier 42 is aligned with an interior cavity in the PCB 82.

FIG. 29 shows the capillaries 15 and 15 inserted into the rest of the calorimetric sensor 10 of FIG. 28.

FIG. 30 is an exploded view of the PCB 82, the calorimetric sensor 12, including the capillaries 14 and 15, and the sensor carrier 42.

In the accompanying drawings, like reference characters refer to the same or similar parts throughout the different views; and apostrophes are used to differentiate multiple instances of the same item or different embodiments of items sharing the same reference numeral. The drawings are not necessarily to scale; instead, an emphasis is placed upon illustrating particular principles in the exemplifications discussed below.

### DETAILED DESCRIPTION

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

Unless otherwise herein defined, used, or characterized, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially (though not perfectly) pure, as practical and imperfect realities may apply; e.g., the potential presence of at least trace impurities (e.g., at less than 1 or 2%) can be understood as being within the scope of the description. Likewise, if a particular shape is referenced, the shape is intended to include imperfect variations from ideal shapes, e.g., due to manufacturing tolerances. Percentages or concentrations expressed herein can be in terms of weight or volume. Processes, procedures, and phenomena described below can occur at ambient pressure (e.g., about 50-120 kPa—for example, about 90-110 kPa) and temperature (e.g., −20 to 50° C.—for example, about 10-35° C.) unless otherwise specified.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments.

Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. The term, “about,” can mean within +10% of the value recited. In addition, where a range of values is provided, each subrange and each individual value between the upper and lower ends of the range is contemplated and therefore disclosed.

Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limited to specifics of the exemplary embodiments. As used herein, singular forms, such as those introduced with the articles, “a” and “an,” are intended to include the plural forms as well, unless the context indicates otherwise. Additionally, the terms, “includes,” “including,” “comprises,” and “comprising,” specify the presence of the stated elements or steps but do not preclude the presence or addition of one or more other elements or steps.

Additionally, the various components identified herein can be provided in an assembled and finished form; or some or all of the components can be packaged together and marketed as a kit with instructions (e.g., in written, video, or audio form) for assembly and/or modification by a customer to produce a finished product.

### Design of Calorimetric Sensor:

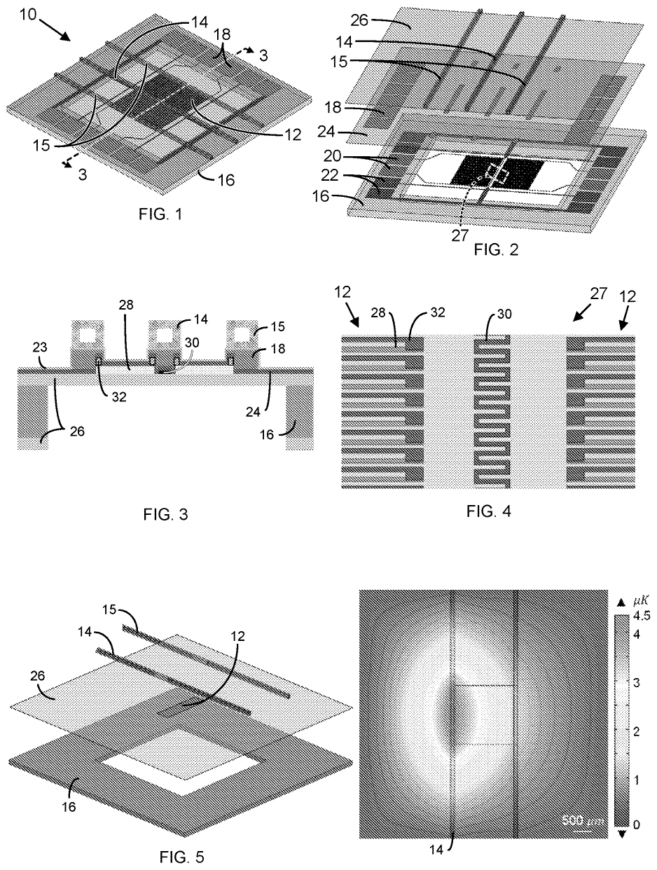

FIGS. 1-4 illustrate a design concept and overall layout of a capillary picocalorimetry sensor 10. Superior resolution of the calorimetric sensor 10 is achieved through high-sensitivity nanofabricated thermopiles 12 [formed, e.g., of nichrome (an alloy of nickel and chromium) 28 and constantan (an alloy of copper and nickel) 32, as shown in FIG. 4] coupled to a set of capillaries (formed, e.g., of a borosilicate glass) 14 and 15 and a low-heat loss environment [provided, e.g., by a thin (e.g., less than 1-μm thick—for example, 600-n thick) thermally insulating membrane 26—e.g., in the form of a low-pressure chemical-vapor-deposited silicon nitride (Si₃N₄) membrane—on which the capillaries 14 and 15 and thermopiles 12 are mounted and by the vacuum atmosphere provided by the chamber in which the calorimetric sensor 10 is mounted]. The heat output of a biological sample is determined by measuring the temperature difference between a central sample capillary 14 that contains the biological sample and two reference capillaries 15 that contain suitable reference fluids on opposite sides of the central sample capillary 14. The temperature measurement is achieved by mounting the capillaries 14 and 15 on a thin silicon nitride membrane 26 that contains low-noise thermopiles 12 that respectively extend between the central sample capillary 14 and the respective reference capillaries 15 on each side of the central sample capillary 14 to provide a direct differential temperature measurement from the central sample capillary 14 to each of the reference capillaries 15. A thin conductive layer, formed, e.g., of gold (Au) 18, is embedded within the silicon nitride membrane 26 to define the sample area and the reference area and to ensure temperature uniformity. The area around the sample capillary 14 has a built-in tungsten (W) heating element 30 embedded in the silicon nitride membrane 26 to directly calibrate the output of the calorimetric sensor 10 to the heat dissipated in the sample area.

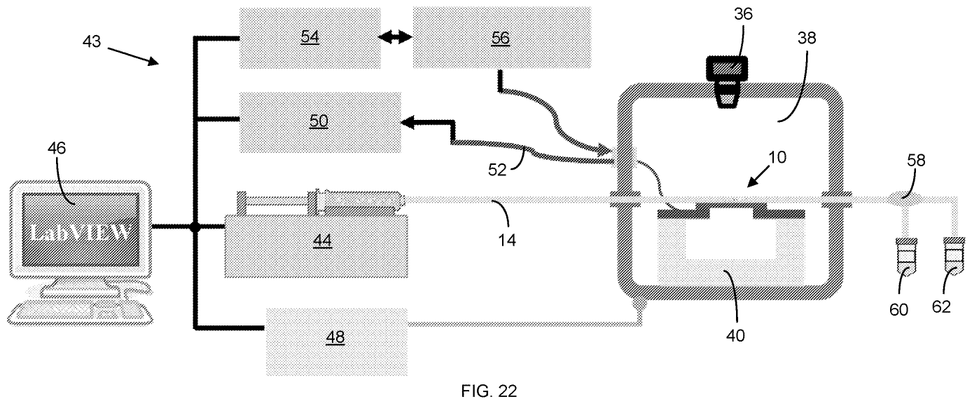

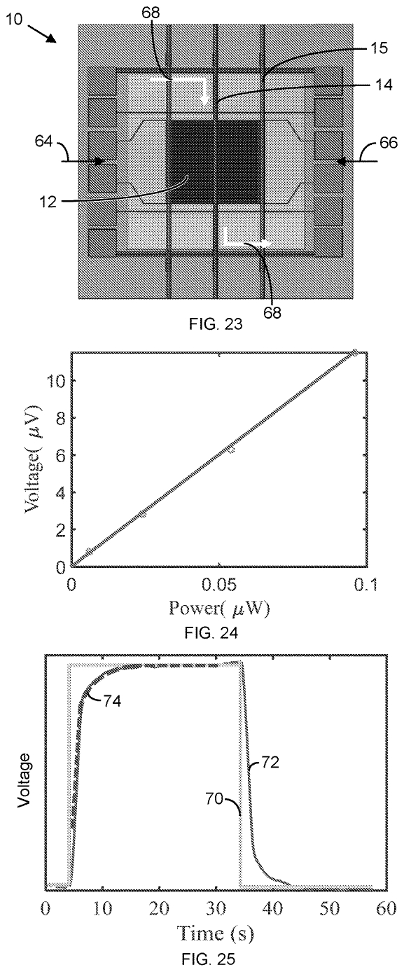

In principle, one reference capillary 15 is sufficient to make a calorimetric measurement. However, using two reference capillaries 15 (particularly when placed on opposite sides of the sample capillary 14) makes it possible to eliminate the effects of minute temperature gradients that may occur inside the measurement system (e.g., by averaging measurements from two reference capillaries 15). This capability is important for both long-term measurements and for measurements of metabolic rates on the order of a few (e.g., 3-5) nW or less. The use of capillaries 14 and 15 can offer the following advantages: 1) the biological samples can be loaded onto the calorimetric sensor 10 using an automated liquid-sample handling system 44 (shown in FIG. 22); 2) the use of capillaries 14 and 15 eliminates sample evaporation providing a cleaner calorimetric signal and increasing the longevity of the biological sample; and 3) the calorimetric sensor 10 can be housed inside a vacuum chamber to minimize heat loss to the environment, thus maximizing the calorimetric signal without adversely affecting the biological sample.

### Performance Simulations:

A detailed three-dimensional thermal model was constructed to predict which parameters play the largest role in calorimetric sensitivity and to quantitatively analyze the temperature distribution within the calorimetric sensor and surrounding area using a commercial finite element package (e.g., COMSOL MULTIPHYSICS simulation software from COMSOL, Inc.). The three-dimensional thermal model considers heat loss by radiation and conduction through the capillaries and the sensor membrane. Heat loss by natural convection is absent because the calorimetric sensor is housed in a vacuum chamber. The thermopiles in the model include a large number of nichrome and constantan thermocouples connected in series [though not shown in FIGS. 1 and 2, the thermocouples are connected out-of-plane along the left and right edges of the calorimetric sensors 10]. The resolution of a given sensor design is then characterized by its noise equivalent power (NEP) and can be written as

$$NEP = \frac{{V}_{JN}}{Σ}$$

[i.e., the ratio of the Johnson noise (V_{JN}) in the thermopile to the thermopile responsivity (Σ)].

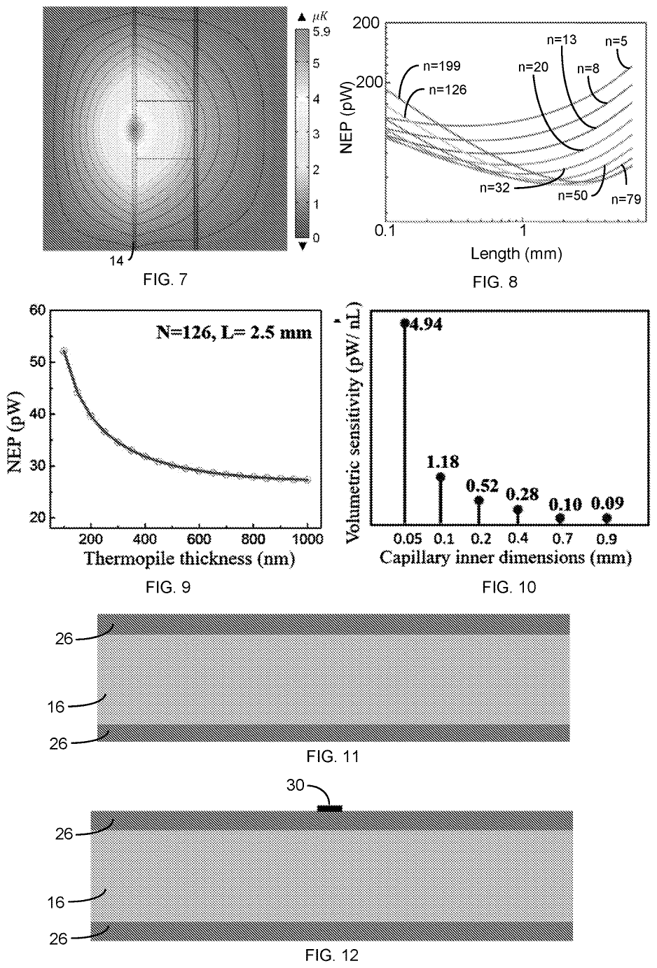

The results of simulations of the configuration shown in exploded form in FIG. 5 are summarized in FIGS. 6-10. FIGS. 6 and 7 show the simulation results for two test cases using this configuration. In one case [the temperature distribution of which is shown in FIG. 6, which is a distributed sample with a noise equivalent power (NEP) of 32 pW], a biological sample (e.g., a suspension of bacteria) is uniformly distributed in the media in the sample capillary 14; and heat is produced throughout the entire sample section, as shown in FIG. 6 by the dark region extending from the sample capillary 14; in the other case [the temperature distribution of which is shown in FIG. 7], a single organism (e.g., an oocyte) produces heat in just one location of the sample volume, as shown by the smaller dark region extending from the sample capillary 14 in FIG. 7. In this case, the NEP was 31 pW. It is evident from FIGS. 6 and 7 that even though the temperature distribution in the latter case is not nearly as uniform as in the former, the sensitivity of the calorimetric sensor is very nearly the same. Consequently, we expect that it will not be necessary to differentiate between the two cases.

As illustrated in FIG. 8, a distinct minimum in the noise equivalent power (NEP) is obtained as a function of thermopile width and length, resulting in an optimum resolution of approximately 32 pW. As shown in FIG. 9, this result can be further improved by increasing the thickness of the thermopile 12, which, in this example, is 2.5-mm long and includes 126 thermocouples in series. To measure biological system with different sample volumes, designs of calorimetric sensors are optimized for a variety of capillary dimensions. The corresponding volumetric sensitivity as a function of capillary dimensions is shown in FIG. 10. It's evident that the size of the capillary 14/15 has a significant effect on the volumetric sensitivity. The projected NEP represents an approximately order of magnitude improvement in resolution compared to existing calorimetric sensors.

### Fabrication of the Sensor:

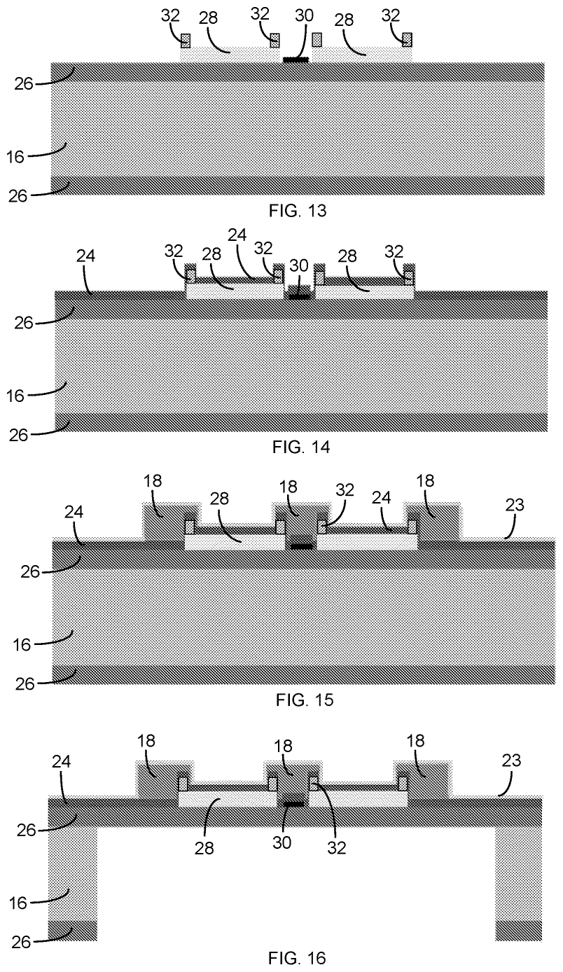

Sensors were fabricated using various microfabrication techniques. First, a 600-nm silicon-nitride coating 26, as shown in FIG. 11, is grown on a silicon substrate 16 using a low-pressure chemical-vapor-deposition (LPCVD) process. Then, at least one tungsten heating element 30 with a thickness of 150 nm, as shown in FIG. 12, and thermopiles formed of nichrome 28 and constantan 32 with a thickness of 500 nm, as shown in FIG. 13, are patterned on top of the silicon-nitride coating 26 using magnetron sputtering and photolithographic lift-off techniques. A layer of 5-nm titanium is deposited prior to deposition of each metal layer to ensure good adhesion. The heating element 30 and thermopiles 12 are subsequently coated with 30 nm of HfO₂ using an atomic-layer-deposition (ALD) process and a thermally and electrically insulating coating in the form of 300 nm of silicon nitride 24 using a plasma-enhanced chemical-vapor-deposition (PECVD) process, as shown in FIG. 14. This electrically insulating (dielectric) layer 24 serves to electrically isolate the heating element 30 and thermopiles 12 and to prevent shorts. Then, 600 nm of conductor (e.g., gold) 18 is sputtered deposited in the sample and reference areas to enhance the temperature uniformity in these areas, followed by 100 nm of PECVD silicon nitride 23 to encapsulate the conductor 18, as shown in FIG. 15. Next, the silicon substrate 16 is selectively etched away, as shown in FIG. 16, using a deep-reactive-ion etch based on the Bosch process to create freestanding sensor membranes extending across the remaining silicon substrate frame spanning the underlying cavity created by the etched removal of the silicon in the internal region of the calorimetric sensor 10.

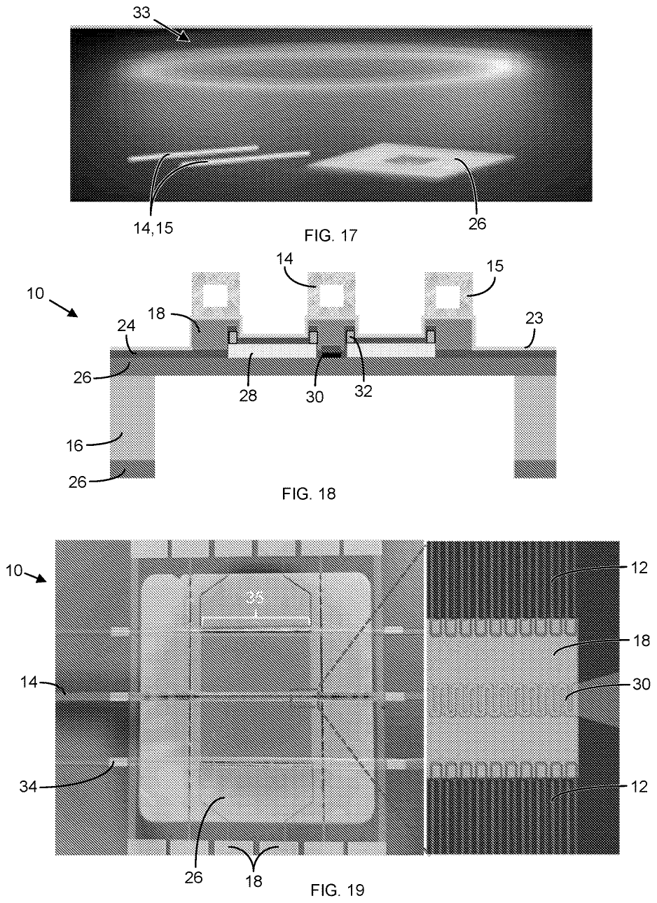

At this point, the sample capillary 14 and reference capillaries 15 are coupled to the rest of the calorimetric sensor 10. Three borosilicate capillary tubes 14 and 15 (100×100 μm², wall thickness of 25 μm) are placed on the remainder of the micromachined calorimetric sensor 10 and aligned with the gold-coated sample and reference areas. For the thermopile 12 to measure as small of a temperature difference as possible, the capillaries 14 and 15 are mounted in good thermal contact with the membrane 26, which is a non-trivial achievement given the fragility of the silicon-nitride membranes 26. We achieved this contact through use of capillary forces. Prior to mounting the capillaries 14 and 15 onto the sensor membrane 26, the calorimetric sensor 10, including the capillaries 14 and 15 are exposed to an oxygen plasma 33 to remove any organic contaminants and to make their surfaces hydrophilic, as shown in FIG. 17.

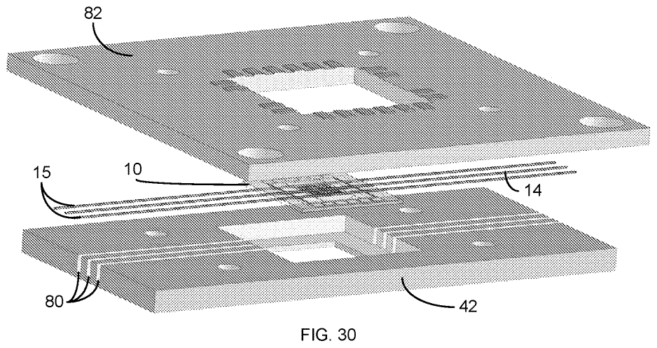

The calorimetric sensor 10, which includes the substrate 16, the thermally and electrically insulating membrane 26, the conductive strips 18, the thermopiles 12, the thermally and electrically insulating coating 24, and capillaries 14, 15 is carried and protected by a 3D-printed sensor carrier 42, as shown in FIG. 27). In the general installing process, the calorimetric sensor 10 is first aligned and glued in the interior cavity of a 3D-printed sensor carrier 42. The sensor carrier 42 is then glued to the backside of a PCB 82, as shown in FIG. 28, prior to wire bonding the calorimetric sensor 10 (without the capillaries) to the PCB 82. The capillaries 14 and 15 are then inserted into the designed grooves 80 of the sensor carrier 42 (from the side), as shown in FIG. 29, and aligned to the sensing areas of the calorimetric sensor 10 under a microscope 36. A droplet of water is then applied to the areas of the thermally and electrically insulating coating 23, 24 (e.g., formed of Si₃N₄) where the capillaries 14 and 15 contact the coating 23, 24. As the water evaporates, capillary action will bring the thermally and electrically insulating coating 23, 24 into contact with the capillaries 14 and 15, creating a permanent bond between them. Finally, the capillaries 14 and 15 are glued to the silicon substrate 16 the sensing assembly and to the sensor carrier 42 to avoid breaking the membrane 26 by potentially moving the capillaries 14 and 15 at the two ends. An exploded view of the assembly is shown in FIG. 30.

### Experimental Setup and Measurement

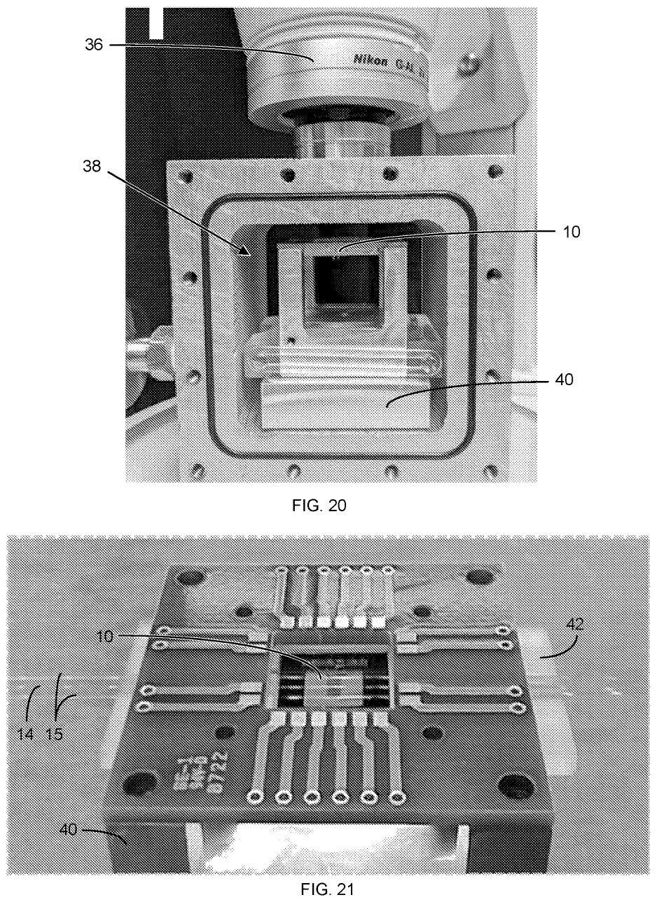

An optical image of a finished calorimeter sensor 10 is shown in FIG. 19. The transparent areas correspond to the freestanding Si₃N₄ membrane 26 that supports the calorimetric sensor 10. The capillaries 14 and 15 were well aligned at the sensing areas of the sample area 35 on the membrane 26 with the aid of alignment marks 34 made of gold on the silicon substrate 16. The sensing area in the center of a sensor membrane 26 is zoomed in and shown in FIG. 19. The gold pad 18, the serpentine heating element 30, and the thermopiles 12 are readily discerned. The calorimetric sensor 10 is housed in a thermally insulated vacuum chamber 38, as shown in FIG. 20, with active temperature control that also serves as a Faraday cage. During operation of the calorimetric sensor 10, the vacuum chamber 38 can create a “vacuum” atmosphere with a reduced gas pressure, e.g., at least an order of magnitude lower than the ambient air pressure. The vacuum chamber 38 is equipped with temperature sensors, heating elements, and proportional-integral-derivative (PID) temperature controllers (e.g., CN16DPT controllers from OMEGA Engineering) to keep the chamber 38 at a uniform fixed temperature and thus minimize any differential radiative heat transfer between the chamber 38 and the sample/reference capillaries 14 and 15. The details as to how the calorimetric sensor 10 is connected to the electrical instruments and installed in the vacuum chamber 38 are shown in FIG. 21.

The method can be executed via automated process commands generated via a computer 46, as shown in FIG. 22, e.g., operating LABVIEW software (from National Instruments Corp.), which is non-transitorily stored on a computer-readable medium in communication with a processor in the communicator, as a graphical programming environment to control the hardware in the system with which it is in communication. The computer also receives operational data (e.g., via an input port in communication with the processor), including the measurements from the thermopiles using a data acquisition (DAQ) system 54, such as a NI DAQ system (from National Instruments Corp.), to determine the bioenergetic level of the biological sample in the sample capillary 14. The DAQ system 54 is electrically coupled with a current source 56 configured to supply electric current to the calorimetric sensor 10. Nanovoltmeters 50 are also electrically coupled with the calorimetric sensor 10 for measuring the detection of voltage therefrom. The DAQ system 54, the nanovoltmeters 50, the sample handler 44, and temperature controls 48 configured to control the temperature in the vacuum chamber 38 are all electrically coupled with the computer 46 to receive instructions therefrom and/or to provide feedback thereto.

The biological samples and reference fluids are loaded into the sample capillary 14 and the reference capillaries 15 of the calorimetric sensor 10 using one or more syringe pumps of a high-throughput sample handler 44, as shown in FIG. 22. When a syringe pump is attached to one end of the sample capillary 14 and the other end of the sample capillary 14 is inserted into a sample collection receptacle 60 in the form of an Eppendorf tube 50, the liquid medium containing the biological sample or the reference fluid, which can have the same composition as the liquid medium minus the biological sample, can be loaded by suction. After the measurement, the sample can be expelled, and the sample capillary 14 rinsed as necessary by cycling the syringe pump. The reference sample can either remain in the reference capillaries 15 or can be replaced via the pump with each test. This simple setup is sufficient to evaluate the performance of the system and to perform a variety of measurements on biological samples where the metabolically active species is uniformly distributed in the sample media (e.g., suspended cells). If measurements need to be made on individual organisms, optical access can be provided for an optical device 36, such as a microscope, as shown in FIG. 20 to the sample capillary to ensure that the organism is located in the sensing area of the pico-calorimetry sensor 10. High-throughput measurements may require more extensive sample handling, which can be accomplished using a commercial autoinjector system. Commercial automated systems are available that can reliably inject 5-nL or greater volume samples.

The calorimetric sensor 10 is calibrated using the built-in heating capability provided by the heating element 30. This approach has the advantage that the input power can be precisely controlled by varying the current through the heating element 30. The responsivity of the calorimetric sensor 10 is then determined directly from the response of the thermopile 12 as a function of input power. In practice, the calorimetric sensors 10 are calibrated by applying a current to the heating element 10 integrated in the sample area 35 using the current source 56 (in this case, a custom-built modified Howland current source, controlled by an NI 9263 voltage output module from National Instruments. The voltage signals from the thermopiles are measured using two two-channel nanovoltmeters (Keithley 2182A voltmeters). FIG. 24 shows the voltage of a thermopile as a function of applied powers at steady state. As shown in FIG. 24, the response of the calorimetric sensors is linear over the entire range of input power and the calibrated responsivity is 120 V/W. The measured resistance of the thermopile of the fabricated sensor shown in FIG. 19 is 94 k (2, which corresponds to a Johnson noise of 5.6 nV and a resolution of 47 pW. The transient behavior of the sensor is shown in FIG. 25, which plots the power voltage of the power pulse 70, the voltage response 72, and a fitting curve 44, and where it is evident from the figure that the response time of a sensor is on the order of a few seconds, which is advantageous for making high-throughput measurements.

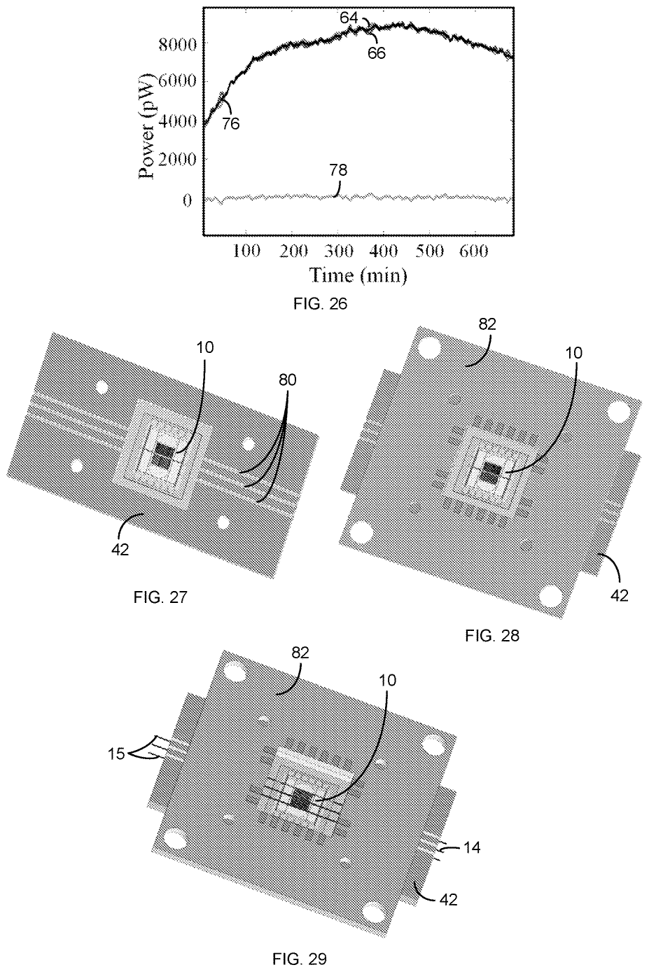

FIG. 26 shows the signals obtained from two individual thermopiles 64 and 66 that are colinear with the sample area over a period of approximately ten hours without any power applied to the heating element 30. Along with these signals, the average 76 and the half difference 78 of the thermopile signals are also shown. The difference signal 78 is proportional to the in-plane temperature gradient in the direction of the thermopiles, while the average 76 of the two thermopiles yields the calorimetric-sensor signal corrected for the in-plane temperature gradient. It is evident from FIG. 26 that the stability of the difference signal 78 is significantly improved compared to the signals from the individual thermopiles 64 and 66 or the average signal 76.

In describing embodiments, herein, specific terminology is used for the sake of clarity. For the purpose of description, specific terms are intended to at least include technical and functional equivalents that operate in a similar manner to accomplish a similar result. Additionally, in some instances where a particular embodiment includes a plurality of system elements or method steps, those elements or steps may be replaced with a single element or step. Likewise, a single element or step may be replaced with a plurality of elements or steps that serve the same purpose. Further, where parameters for various properties or other values are specified herein for embodiments, those parameters or values can be adjusted up or down by 1/100^{th}, 1/50^{th}, 1/20^{th}, 1/10^{th}, ⅕^{th}, ⅓^{rd}, ½, ⅔^{rd}, ¾^{th}, ⅘^{th}, 9/10^{th}, 19/20^{th}, 49/50^{th}, 99/100^{th}, etc. (or up by a factor of 1, 2, 3, 4, 5, 6, 8, 10, 20, 50, 100, etc.), or by rounded-off approximations thereof or within a range of the specified parameter up to or down to any of the variations specified above (e.g., for a specified parameter of 100 and a variation of 1/100^{th}, the value of the parameter may be in a range from 0.99 to 1.01), unless otherwise specified. Further still, where methods are recited and where steps/stages are recited in a particular order—with or without sequenced prefacing characters added for ease of reference—the steps/stages are not to be interpreted as being temporally limited to the order in which they are recited unless otherwise specified or implied by the terms and phrasing.

Additional Examples Consistent with the Present Teachings are Set Out in the Following Numbered Clauses:
1. A calorimetric sensor for high-throughput screening of bioenergetics, comprising:
   a substrate defining an interior cavity;
   a thermally and electrically insulating membrane extending from the substrate across the interior cavity;
   a plurality of thermally conductive strips bonded to the thermally insulating membrane;
   a first thermopile on the thermally and electrically insulating membrane and comprising a plurality of thermocouples connected in series, wherein the thermocouples of the first thermopile extend between the first thermally conductive strip and the second thermally conductive strip and provide thermal communication therebetween and are configured to provide a measurement of a temperature difference between the first and second thermally conductive strips;
   a thermally and electrically insulating coating on the thermally and electrically insulating membrane, the thermally conductive strips, and the first thermopile, wherein these components, together, form a sensing assembly;
   a sample capillary with a hydrophilic surface adhered to the thermally and electrically insulating coating opposite a first of the thermally conductive strips via application of water at an interface of the sample capillary and the thermally and electrically insulating coating and via capillary action during evaporation of the water; and
   a first reference capillary with a hydrophilic surface adhered to the thermally and electrically insulating coating opposite a second of the thermally conductive strips via application of water at an interface of the reference capillary and the thermally and electrically insulating coating and via capillary action during evaporation of the water.
2. The calorimetric sensor of clause 1, wherein the calorimetric sensor is mounted in a vacuum chamber.
3. The calorimetric sensor of clause 1 or 2, wherein the thermally and electrically insulating membrane and the thermally and electrically insulating coating comprise silicon nitride.
4. The calorimetric sensor of any of clauses 1-3, wherein the thermally conductive strips comprise gold.
5. The calorimetric sensor of any of clauses 1-4, wherein the capillaries comprise a borosilicate glass.
6. The calorimetric sensor of any of clauses 1-5, wherein the thermopile comprises nichrome and constantan.
7. The calorimetric sensor of any of clauses 1-6, further comprising:
   a second reference capillary with a hydrophilic surface adhered to the thermally and electrically insulating coating opposite a third of the thermally conductive strips via application of water at an interface of the reference capillary and the thermally and electrically insulating coating and via capillary action during evaporation of the water, wherein the first thermally conductive strip is between the second and third thermally conductive strips; and
   a second thermopile on the thermally and electrically insulating membrane on an opposite side of the first thermally conductive strip from the first thermopile, wherein the second thermopile also comprises a plurality of thermocouples connected in series, wherein the thermocouples of the second thermopile extend between the first thermally conductive strip and the third thermally conductive strip and are configured to provide a measurement of a temperature difference between the first and third thermally conductive strips.

8. The calorimetric sensor of any of clauses 1-7, further comprising a heating element on the thermally and electrically insulating membrane between the thermally and electrically insulating membrane and the sample capillary.
9. The calorimetric sensor of any of clauses 1-8, wherein the calorimetric sensor has dimensions of less than 1 cm.
10. A method for performing calorimetry on a biological sample, comprising;
   using the calorimetric sensor of any of clauses 1-9, passing a biological sample through the sample capillary;
   filling the first reference capillary with a reference fluid;
   using the first thermopile to measure a temperature difference from the first conductive strip to the second conductive strip; and
   determining a bioenergetic level of the biological sample based on the measurement of the temperature difference from the first conductive strip to the second conductive strip.
11. The method of clause 10, wherein the biological sample is a single cell.
12. The method of clause 11, wherein the cell is a bacterium.
13. The method of clause 12, further comprising exposing the bacterium to an antibiotic before passing the bacterium through the sample capillary.
14. The method of clause 13, further comprising repeating the method over a plurality of iterations and changing at least one of (a) the antibiotic or (b) a dosage of the antibiotic in different iterations of the method.
15. The method of clause 14, further comprising determining an optimized antibiotic treatment for a patient based on a comparison of the measurements of the bioenergetic levels of the bacteria in the different iterations of the method.
16. The method of any of clauses 10-15, wherein the calorimetric sensor is mounted in a vacuum chamber, the method further comprising providing a vacuum atmosphere in the vacuum chamber while the method is practiced.
17. The method of any of clauses 10-16, using the calorimetric sensor of clause 7, the method further comprising:
   filling the second reference capillary with additional reference fluid;
   using the second thermopile to measure a temperature difference from the first conductive strip to the third conductive strip;
   determining at least one of (a) an average of and (b) a difference between the temperature measurements of the first and second thermopiles to calibrate for a temperature gradient across the calorimetric sensor; and
   determining a bioenergetic level of the biological sample based on the determination of the previous step.
18. The method of any of clauses 10-16, using the calorimetric sensor of clause 8, the method further comprising:
   heating the heating element before or after the method of clause 10 is performed; and
   measuring the temperature difference across the first thermopile while the heating element is heated to then calibrate the measurements of the temperature difference with the biological sample passing through the sample capillary.
19. A method for fabricating a calorimetric sensor for high-throughput screening of bioenergetics, comprising:
   depositing a thermally and electrically insulating membrane onto a substrate;
   etching a central region of the substrate to create an interior cavity;
   forming at least one thermopile on the thermally and electrically insulating membrane;
   depositing a plurality of thermally conductive strips on the thermally and electrically insulating membrane, wherein each thermally conductive strip is in thermal contact with at least one of the thermopiles;
   depositing a thermally and electrically insulating coating on the thermally and electrically insulating membrane, the thermally conductive strips, and the at least one thermopile, wherein these components, together, form a sensing assembly;
   providing a surface treatment to a sample capillary and at least one reference capillary to give the capillaries hydrophilic surfaces;
   providing the surface treatment to the thermally and electrically insulating coating to give the thermally and electrically insulating coating a hydrophilic surface; then
   placing the sample capillary and the at least one reference capillary in contact with the thermally and electrically insulating coating opposite respective thermally conductive strips; and
   applying water at interfaces of the capillaries and the thermally and electrically insulating coating and evaporating the water to adhere the capillaries to the thermally and electrically insulating coating via capillary action.
20. The method of clause 19, wherein the surface treatment includes exposing the capillaries to an oxygen plasma.
21. The method of clause 19 or 20, wherein two reference capillaries are applied to and adhered to the thermally and electrically insulating coating on opposite sides of the sample capillary.

While this invention has been shown and described with references to particular embodiments thereof, those skilled in the art will understand that various substitutions and alterations in form and details may be made therein without departing from the scope of the invention. Further still, other aspects, functions, and advantages are also within the scope of the invention; and all embodiments of the invention need not necessarily achieve all of the advantages or possess all of the characteristics described above. Additionally, steps, elements, and features discussed herein in connection with one embodiment can likewise be used in conjunction with other embodiments. The contents of references, including reference texts, journal articles, patents, patent applications, etc., cited throughout the text are hereby incorporated by reference in their entirety for all purposes; and all appropriate combinations of embodiments, features, characterizations, and methods from these references and the present disclosure may be included in embodiments of this invention. Still further, the components and steps identified in the Background section are integral to this disclosure and can be used in conjunction with or substituted for components and steps described elsewhere in the disclosure within the scope of the invention.

What is claimed is:

1. A calorimetric sensor for high-throughput screening of bioenergetics, comprising:
a substrate defining an interior cavity;
a thermally and electrically insulating membrane extending from the substrate across the interior cavity;
a plurality of thermally conductive strips bonded to the thermally and electrically insulating membrane;
a first thermopile on the thermally and electrically insulating membrane and comprising a plurality of thermocouples connected in series, wherein the thermocouples of the first thermopile extend between the first thermally conductive strip and the second thermally conductive strip and provide thermal communication therebetween and are configured to provide a measurement of a temperature difference between the first and second thermally conductive strips;
a thermally and electrically insulating coating on the thermally and electrically insulating membrane, the thermally conductive strips, and the first thermopile, wherein these components, together, form a sensing assembly;
a sample capillary with a hydrophilic surface adhered to the thermally and electrically insulating coating opposite a first of the thermally conductive strips via application of water at an interface of the sample capillary and the thermally and electrically insulating coating and via capillary action during evaporation of the water; and
a first reference capillary with a hydrophilic surface adhered to the thermally and electrically insulating coating opposite a second of the thermally conductive strips via application of water at an interface of the reference capillary and the thermally and electrically insulating coating and via capillary action during evaporation of the water.

2. The calorimetric sensor of claim 1, wherein the calorimetric sensor is mounted in a vacuum chamber.

3. The calorimetric sensor of claim 1, wherein the thermally and electrically insulating membrane and the thermally and electrically insulating coating comprise silicon nitride.

4. The calorimetric sensor of claim 1, wherein the thermally conductive strips comprise gold.

5. The calorimetric sensor of claim 1, wherein the capillaries comprise a borosilicate glass.

6. The calorimetric sensor of claim 1, wherein the thermopile comprises nichrome and constantan.

7. The calorimetric sensor of claim 1, further comprising:
a second reference capillary with a hydrophilic surface adhered to the thermally and electrically insulating coating opposite a third of the thermally conductive strips via application of water at an interface of the reference capillary and the thermally and electrically insulating coating and via capillary action during evaporation of the water, wherein the first thermally conductive strip is between the second and third thermally conductive strips; and
a second thermopile on the thermally and electrically insulating membrane on an opposite side of the first thermally conductive strip from the first thermopile, wherein the second thermopile also comprises a plurality of thermocouples connected in series, wherein the thermocouples of the second thermopile extend between the first thermally conductive strip and the third thermally conductive strip and are configured to provide a measurement of a temperature difference between the first and third thermally conductive strips.

8. The calorimetric sensor of claim 1, further comprising a heating element on the thermally and electrically insulating membrane between the thermally and electrically insulating membrane and the sample capillary.

9. The calorimetric sensor of claim 1, wherein the calorimetric sensor has dimensions of less than 1 cm.

10. A method for performing calorimetry on a biological sample, comprising;
using the calorimetric sensor of claim 1, passing a biological sample through the sample capillary;
filling the first reference capillary with a reference fluid;
using the first thermopile to measure a temperature difference from the first conductive strip to the second conductive strip; and
determining a bioenergetic level of the biological sample based on the measurement of the temperature difference from the first conductive strip to the second conductive strip.

11. The method of claim 10, wherein the biological sample is a single cell.

12. The method of claim 11, wherein the cell is a bacterium.

13. The method of claim 12, further comprising exposing the bacterium to an antibiotic before passing the bacterium through the sample capillary.

14. The method of claim 13, further comprising repeating the method over a plurality of iterations and changing at least one of (a) the antibiotic or (b) a dosage of the antibiotic in different iterations of the method.

15. The method of claim 14, further comprising determining an optimized antibiotic treatment for a patient based on a comparison of the measurements of the bioenergetic levels of the bacteria in the different iterations of the method.

16. The method of claim 10, wherein the calorimetric sensor is mounted in a vacuum chamber, the method further comprising providing a vacuum atmosphere in the vacuum chamber while the method is practiced.

17. The method of claim 10, using the calorimetric sensor of claim 7, the method further comprising:
filling the second reference capillary with additional reference fluid;
using the second thermopile to measure a temperature difference from the first conductive strip to the third conductive strip;
determining at least one of (a) an average of and (b) a difference between the temperature measurements of the first and second thermopiles to calibrate for a temperature gradient across the calorimetric sensor; and
determining a bioenergetic level of the biological sample based on the determination of the previous step.

18. The method of claim 10, using the calorimetric sensor of claim 8, the method further comprising:
heating the heating element before or after the method of claim 10 is performed; and
measuring the temperature difference across the first thermopile while the heating element is heated to then calibrate the measurements of the temperature difference with the biological sample passing through the sample capillary.

19. A method for fabricating a calorimetric sensor for high-throughput screening of bioenergetics, comprising:
depositing a thermally and electrically insulating membrane onto a substrate;
etching a central region of the substrate to create an interior cavity;
forming at least one thermopile on the thermally and electrically insulating membrane;
depositing a plurality of thermally conductive strips on the thermally and electrically insulating membrane, wherein each thermally conductive strip is in thermal contact with at least one of the thermopiles;
depositing a thermally and electrically insulating coating on the thermally and electrically insulating membrane, the thermally conductive strips, and the at least one thermopile, wherein these components, together, form a sensing assembly;
providing a surface treatment to a sample capillary and at least one reference capillary to give the capillaries hydrophilic surfaces;
providing the surface treatment to the thermally and electrically insulating coating to give the thermally and electrically insulating coating a hydrophilic surface; then placing the sample capillary and the at least one reference capillary in contact with the thermally and electrically insulating coating opposite respective thermally conductive strips; and
applying water at interfaces of the capillaries and the thermally and electrically insulating coating and evaporating the water to adhere the capillaries to the thermally and electrically insulating coating via capillary action.

20. The method of claim 19, wherein the surface treatment includes exposing the capillaries to an oxygen plasma.

21. The method of claim 19, wherein two reference capillaries are applied to and adhered to the thermally and electrically insulating coating on opposite sides of the sample capillary.
